(19) United States
(12) Patent Application Publication
(10) Pub. No.: US 2016/0120771 A1
(43) Pub. Date: May 5, 2016

(54) COSMETIC COMPOSITION COMPRISING AN AQUEOUS PHASE AND A FATTY PHASE THAT ARE VISUALLY DISTINCT

(71) Applicant: L'OREAL, Paris (FR)

(72) Inventors: Frédéric SIMONET, Clichy (FR); Sylvain KRAVTCHENKO, Sigogne (FR); Aldo PIZZINO, Saint-Ouen (FR); Gérard GABIN, Paris (FR); Xavier JOSEPH, Paris (FR); Sandrine DECOSTER, Saint-Gratien (FR)

(21) Appl. No.: 14/893,142

(22) PCT Filed: May 26, 2014
(86) PCT No.: PCT/EP2014/060862

§ 371 Date: Nov. 23, 2015

(30) Foreign Application Priority Data
May 24, 2013 (FR) 1354689
May 23, 2014 (FR) 1454669

(51) Int. Cl. A61K 8/03; A61Q 5/00; A61K 8/04; A45D 7/06; A61K 8/92; A61K 8/58; A45D 7/04; A61Q 5/06; A61K 8/81

(52) U.S. Cl. CPC A61K 8/03; A61Q 5/06; A61Q 5/00; A61K 8/042; A61K 8/8147; A61K 8/922; A61K 8/58; A61K 8/8152; A45D 7/04; A45D 7/06

(57) ABSTRACT

The present invention relates to a cosmetic composition comprising: —a fatty phase comprising at least one oil; —an aqueous phase comprising at least one aqueous-phase thickener, the two phases being visually distinct.

The present invention relates to a cosmetic composition comprising a fatty phase and an aqueous phase that are visually distinct, the aqueous phase comprising at least one aqueous-phase thickener and the fatty phase comprising at least one oil, and to a hair treatment process comprising the application of the composition.

In the field of styling, in particular among hair products intended for shaping and/or holding the hairstyle, hair compositions are generally in the form of hair gels, lotions, mousses or sprays.

In particular, hair gels allow good hold of the head of hair. However, many hair gels do not give the hair sufficient cosmeticity and the final result lacks a natural look.

In addition, the majority of hair gels have a uniform appearance that is not very appealing.

It has already been proposed to make compositions comprising visually distinct phases, as described, for example, in patent applications WO 2006/093 742, WO 2006/042 179, WO 2006/010 090 or WO 2007/004 200. In practice, the weight ratio between the various phases can only generally vary within a limited range of values and the preparation of these compositions is often difficult, for a stability of the compositions obtained that is not entirely satisfactory.

Haircare products often use conditioning agents, especially oils. Anhydrous compositions containing oils very often lead to hair that feels too greasy. In aqueous compositions, a limitation is very often posed by the very low solubility of oils in water, generally imposing the use of surfactants in aqueous compositions containing these oils, often with, as a corollary, an opaque final appearance of creamy emulsion type.

There is thus a real need to find cosmetic compositions, which afford haircare and which allow easy formulation of oils in aqueous medium.

There is also a need to find styling compositions which allow good hairstyle hold while at the same time giving the hair cosmeticity.

There is also a need to find cosmetic compositions, especially for styling, which make it possible to obtain a hairstyle with a natural look.

There is also a need to find cosmetic compositions that have a novel, more attractive aesthetic look.

The Applicant has discovered that the choice of an aqueous phase comprising at least one aqueous-phase thickener and of a fatty phase comprising at least one oil, in which the two phases are visually distinct, makes it possible to satisfy at least one of these needs.

Thus, one subject of the present invention is a cosmetic composition comprising:
a fatty phase comprising at least one oil;
an aqueous phase comprising at least one aqueous-phase thickener, the two phases being visually distinct.

The present invention makes it possible to prepare aesthetic compositions that care for the hair.

It is thus possible to obtain a composition which comprises a relatively large amount of oil.

A subject of the invention is also the use of the said composition for caring for the hair and/or for shaping the hair.

In the text hereinbelow, the expression “at least one” is equivalent to “one or more” and, unless otherwise indicated, the limits of a range of values are included in that range.

The composition according to the invention comprises two visually distinct phases. The term “two visually distinct phases” means that the phases may be distinguished from each other by a person's naked eye, unlike phases forming emulsions or dispersions of homogeneous particles. Preferably, at least one of the phases occupies zones forming volutes or marbling, preferably more than 1 cm in length. Preferably, one of the phases is not in the form of globules. More preferably, none of the phases is in the form of globules.

The two phases are visually distinct in a stable manner, which means that the areas occupied by the two phases do not move in response to a simple inversion of the container which contains them, without any other stress applied to the composition. The two phases are not able to mix when you shake the container that contains them. The two phases are particularly not a bi-phase liquid for which two phases occupy separate areas one above the other and which, when the container returns, mix.

According to a preferred embodiment, the composition according to the invention is such that it has a yield stress greater than or equal to 0.1 Pa.

Preferably, the composition according to the invention is such that it has a yield stress ranging from 0.1 Pa to 300 Pa, preferably ranging from 1 Pa to 250 Pa, and still preferably from 10 Pa to 200 Pa.

Preferably, each phase has a yield stress at 25° C. of from 0.1 Pa to 300 Pa, preferably 1 Pa to 250 Pa, and more preferably from 10 Pa to 200 Pa.

The yield stress is determined by stress sweep at 25° C. A stress controlled rotating rheometer, Thermo Haake RS600, with a sand blasted cone and plate geometry is used. The temperature is controlled by a Peltier plate and an anti-evaporation device (solvent trap filled with water for measurement at 25° C.).

A logarithmic ramp of 0.5 to 500 Pa is carried out over a period of 3 minutes. Two linear fitting curves corresponding to stationary regime (solid and liquid behavior) are plotted on the curve representing the deformation function of stress (logarithmic). The intersection of these two linear curves gives the value of the yield stress.

To measure the yield stress of each phase, the measurement is carried out before mixing the two phases.

As mentioned previously, the composition according to the invention comprises a fatty phase.

The fatty phase of the composition in accordance with the invention comprises at least one oil.

The term “oil” means any fatty substance that is in liquid form at room temperature (25° C.) and at atmospheric pressure.

The oil(s) present in the composition may be volatile or non-volatile.

The volatile or non-volatile oils may be hydrocarbon-based oils, in particular of animal or plant origin, synthetic oils, silicone oils or fluoro oils, or mixtures thereof.

For the purposes of the present invention, the term “silicone oil” means an oil comprising at least one silicon atom, and in particular at least one Si—O group.

The term “hydrocarbon-based oil” means an oil mainly containing hydrogen and carbon atoms, and optionally oxygen, nitrogen, sulfur and/or phosphorus atoms. A hydrocarbon-based oil does not comprise any silicon atoms.

Non-Volatile Oils

For the purposes of the present invention, the term “non-volatile oil” means an oil having a vapour pressure of less than 0.13 Pa (0.01 mmHg).

The non-volatile oils may be chosen in particular from non-volatile hydrocarbon-based oils, which may be fluorinated, and/or non-volatile silicone oils.

As non-volatile hydrocarbon-based oils that are suitable for use in the invention, mention may be made in particular of:

hydrocarbon-based oils of animal origin;

hydrocarbon-based oils of plant origin such as phytostearyl esters, such as phytostearyl oleate, phytostearyl isostearate and lauroyl/octyldodecyl/phytostearyl glutamate, for example sold under the name Eldew PS203 by Ajinomoto, triglycerides consisting of fatty acid esters of glycerol, the fatty acids of which may have chain lengths ranging from C4 to C24, these chains possibly being linear or branched, and saturated or unsaturated; these oils are especially heptanoic or octanoic triglycerides, sweet almond oil, argan oil, avocado oil, groundnut oil, camellia oil, safflower oil, beauty-leaf oil, rapeseed oil, copra oil, coriander oil, marrow oil, wheatgerm oil, jojoba oil or liquid jojoba wax, linseed oil, macadamia oil, corn germ oil, hazelnut oil, walnut oil, vernonia oil, apricot kernel oil, olive oil, evening primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, Brazil nut oil; or alternatively caprylic/capric acid triglycerides, for instance those sold by the company Stearineries Dubois or those sold under the names Miglyol 8100, 812® and 818® by the company Dynamit Nobel, and the refined plant perhydrosqualene sold under the name Fitoderm by the company Cognis; Hydrocarbon-based oils of plant origin are preferably chosen from avocado oil, olive oil, sunflower oil, pracaxi oil, argan oil, camelina oil, jojoba oil and sweet almond oil.

hydrocarbon-based oils of mineral or synthetic origin, for instance:
synthetic ethers containing from 10 to 40 carbon atoms;
linear or branched hydrocarbons of mineral or synthetic origin, such as petroleum jelly, polydecenes, hydrogenated polyisobutene such as Parleam, and squalane, and mixtures thereof, and in particular hydrogenated polyisobutene;
synthetic esters, for instance oils of formula R₁COOR₂ in which R₁ represents a linear or branched fatty acid residue containing from 1 to 40 carbon atoms and R₂ represents a hydrocarbon-based chain that is especially branched, containing from 1 to 40 carbon atoms provided that R₁+R₂ 10.
   The esters may be chosen especially from esters, especially fatty acid esters, for instance:

cetostearyl octanoate, isopropyl alcohol esters, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, for instance isostearyl lactate, octyl hydroxystearate, diisopropyl adipate, heptanoates, and especially isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate, 2-ethylhexyl palmitate, alkyl benzoates, polyethylene glycol diheptanoate, propylene glycol 2-diethylhexanoate, and mixtures thereof, C₁₂-C₁₅ alcohol benzoates, hexyl laurate, neopentanoic acid esters, for instance isodecyl neopentanoate, isotridecyl neopentanoate, isostearyl neopentanoate, octyldodecyl neopentanoate, isononanoic acid esters, for instance isononyl isononanoate, isotridecyl isononanoate, octyl isononanoate, hydroxylated esters, for instance isostearyl lactate and diisostearyl malate;

polyol esters and pentaerythritol esters, for instance dipentaerythrityl tetrahydroxystearate/tetraisostearate;

esters of diol dimers and of diacid dimers, such as Lusplan DD-DA5® and Lusplan DD-DA7® sold by the company Nippon Fine Chemical and described in patent application FR 03/02809;

fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol,

non-salified higher fatty acids such as oleic acid, linoleic acid and linolenic acid, and mixtures thereof, and

dialkyl carbonates, the two alkyl chains possibly being identical or different, such as the dicaprylyl carbonate sold under the name Cetiol CC® by Cognis,

and mixtures thereof.

The non-volatile silicone oils are chosen, for example, from non-volatile polydimethylsiloxanes (PDMSs), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, and dimethicones or phenyl trimethicones with a viscosity of less than or equal to 100 cSt, and mixtures thereof.

The non-volatile oils may be chosen from mixtures of hydrocarbon-based and silicone non-volatile oils.

Volatile Oils

For the purposes of the present invention, the term “volatile oil” means an oil (or non-aqueous medium) that is capable of evaporating on contact with the skin in less than one hour, at room temperature and at atmospheric pressure. The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile hydrocarbon-based oils may be chosen from hydrocarbon-based oils containing from 8 to 16 carbon atoms, and in particular branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

Volatile fluoro oils such as nonafluoromethoxybutane or perfluoromethylcyclopentane, and mixtures thereof, may also be used.

Volatile oils that may also be used include volatile silicones, for instance volatile linear or cyclic silicone oils, especially those with a viscosity ≦8 centistokes (8×10⁻⁶ m²/s), and especially containing from 2 to 10 silicon atoms and in particular from 2 to 7 silicon atoms, these silicones optionally comprising alkyl or alkoxy groups containing from 1 to 10 carbon atoms. As volatile silicone oils that may be used in the invention, mention may be made in particular of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

It is also possible to use a mixture of hydrocarbon-based and silicone volatile oils.

The oil(s) are preferably chosen from C₆-C₁₆ lower alkanes; linear or branched hydrocarbons of mineral or synthetic origin containing more than 16 carbon atoms; non-silicone oils of animal origin or oils of plant origin; fluoro oils; liquid fatty alcohols; liquid fatty esters; non-salified liquid fatty acids; silicone oils; or mixtures thereof, and are preferably chosen from C₆-C₁₆ lower alkanes; linear or branched hydrocarbons of mineral or synthetic origin containing more than 16 carbon atoms; liquid fatty alcohols; oils of plant origin; or mixtures thereof.

The oil is preferably present in a content ranging from 0.1% to 20%, more preferentially in an amount ranging from 1% to 10%, and better still in an amount ranging from 2% to 5% by weight, relative to the total weight of the composition.

The fatty phase of the composition may also comprise one or more fatty-phase thickener(s) and especially oils.

According to the present invention, the term “fatty-phase thickener” means compounds which, by their presence, increase the viscosity of the fatty phase into which they are introduced by at least 20 cps and preferably by at least 50 cps, at 25° C. and at a shear rate of 1 s⁻¹ (the viscosity may be measured using a cone/plate viscometer, a Haake R600 rheometer or the like).

The notion of a fatty-phase thickener is analogous to the notion of a lipophilic thickener.

The fatty-phase thickener(s) used in the composition according to the invention may be mineral or organic.

The mineral fatty-phase thickeners that may be used in the composition according to the invention are preferably mineral particles consisting essentially of mineral oxides and/or hydroxides.

These particles are preferably insoluble in water at room temperature (25° C.). The term “insoluble” means a solubility of less than 0.5% by weight.

Preferably, the number-average primary size of these mineral particles ranges from 0.01 to 500 μm, it preferably ranges from 0.1 to 200 μm and even more preferentially it ranges from 1 to 100 μm.

For the purposes of the present invention, the term “primary particle size” means the maximum dimension that it is possible to measure between two diametrically opposite points on an individual particle.

The size of the mineral particles may be determined by transmission electron microscopy or by measuring the specific surface area via the BET method or by laser granulometry.

The mineral particles that may be used in accordance with the invention may be in various forms, for example in the form of spheres, needles, flakes or platelets.

In a preferred variant of the invention, the mineral fatty-phase thickener(s) are platelet-shaped particles.

The mineral fatty-phase thickener(s) that may be used in the cosmetic composition according to the invention may preferably be chosen from silicas and silicates.

The silicates of the invention may be natural or chemically modified (or synthetic).

Silicates correspond to optionally hydrated silica in which some of the silicon atoms are replaced with metal cations such as Al³⁺, B³⁺, Fe³⁺, Ga³⁺, Be²⁺, Zn²⁺, Mg²⁺, Co³⁺, Ni³⁺, Na⁺, Li⁺, Ca²⁺, Cu²⁺.

More particularly, the silicates that may be used in the context of the invention are chosen from clays of the smectite family such as montmorillonites, hectorites, bentonites, beidellites and saponites, and also of the vermiculite, stevensite and chlorite families.

These clays may be of natural or synthetic origin. Clays that are cosmetically compatible and acceptable with keratin materials are preferably used.

The silicate may be chosen from montmorillonite, bentonite, hectorite, attapulgite and sepiolite, and mixtures thereof.

Mention may thus be made of the compounds sold by the company Laporte under the name Laponite XLG and Laponite XLS.

The silicate(s) are preferably chosen from bentonites and hectorites.

The silicate(s) may be modified with a compound chosen from quaternary amines, tertiary amines, amine acetates, imidazolines, amine soaps, fatty sulfates, alkylarylsulfonates and amine oxides, and mixtures thereof.

As silicates that may be suitable for use, mention may be made of quaternium-18 bentonites such as those sold under the names Bentone 3, Bentone 38 and Bentone 38V by the company Rheox, Tixogel VP by the company United Catalyst, Claytone 34, Claytone 40 and Claytone XL by the company Southern Clay; stearalkonium bentonites such as those sold under the names Bentone 27 by the company Rheox, Tixogel LG by the company United Catalyst and Claytone AF and Claytone APA by the company Southern Clay; quaternium-18/benzalkonium bentonites such as those sold under the names Claytone HT and Claytone PS by the company Southern Clay; quaternium-18 hectorites such as those sold under the names Bentone Gel DOA, Bentone Gel ECO5, Bentone Gel EUG, Bentone Gel IPP, Bentone Gel ISD, Bentone Gel SS71, Bentone Gel VS8 and Bentone Gel VS38 by the company Rheox, and Simagel M and Simagel SI 345 by the company Biophil.

The silicates that may be used in the composition according to the invention may be chosen, in particular, from modified hectorites such as hectorite modified with a C₁₀-C₁₂ fatty acid ammonium chloride, especially distearyldimethylammonium chloride and stearylbenzyldimethylammonium chloride.

As explained previously, the mineral fatty-phase thickener(s) that may be used in the composition according to the invention may be silicas.

The silicas that may be used in the composition according to the invention are preferably fumed silicas.

Fumed silicas may be obtained by high-temperature hydrolysis of a volatile silicon compound in an oxyhydrogen flame, producing a finely divided silica. This process makes it possible especially to obtain hydrophilic silicas which bear a large number of silanol groups at their surface. Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

It is possible to chemically modify the surface of the said silicas, via a chemical reaction generating a reduction in the number of silanol groups. It is possible in particular to replace silanol groups with hydrophobic groups: a hydrophobic silica is then obtained.

The hydrophobic groups may be:

(a) trimethylsiloxyl groups, which are obtained especially by treating fumed silica in the presence of hexamethyldisilazane. Silicas thus treated are known as “Silica silylate” according to the CTFA (6th edition, 1995). They are sold, for example, under the references Aerosil R812® by the company Degussa and Cab-O-Sil TS-530® by the company Cabot;

(b) dimethylsilyloxyl or polydimethylsiloxane groups, which are obtained in particular by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972® and Aerosil R974® by the company Degussa and Cab-O-Sil TS-610® and Cab-O-Sil TS-720® by the company Cabot.

Preferably, the fumed silicas that may be used in the composition according to the invention are hydrophilic, such as the product sold under the name Aerosil 200®.

Preferably, the mineral fatty-phase thickener(s) are chosen from organophilic clays and hydrophilic fumed silicas, and mixtures thereof.

More preferentially, the mineral fatty-phase thickeners are chosen from hectorites modified with a C₁₀-C₁₂ fatty acid ammonium chloride, especially distearyldimethylammonium chloride and stearylbenzyldimethylammonium chloride, and hydrophilic fumed silicas such as the hydrophilic silicas sold under the name Aerosil 200®.

Even more preferentially, the mineral fatty-phase thickeners are chosen from hectorites modified with a C₁₀-C₁₂ fatty acid ammonium chloride, especially hectorite modified with distearyldimethylammonium chloride, such as the product sold under the name Bentone 38VCG by the company Elementis, and the hectorite modified with stearylbenzyldimethylammonium chloride, such as the product sold under the name Bentone 27V by the company Elementis.

As explained previously, the fatty-phase thickener(s) that may be used in the composition according to the invention may also be chosen from organic fatty-phase thickeners.

The organic fatty-phase thickener(s) may be chosen especially from semicrystalline polymers, non-silicone polyamides, silicone polyamides, monoalkyl or polyalkyl esters of saccharides or of polysaccharides, N-acylamino acid amide derivatives, copolymers comprising an alkylene and/or styrene block, and elastomeric organopolysiloxanes, and mixtures thereof. These copolymers may be diblock, triblock or multi-block polymers, radial-block polymers, also known as star copolymers, or alternatively comb polymers.

Preferably, the fatty-phase thickener(s) are chosen from mineral thickeners.

More preferably, the fatty-phase thickener(s) are chosen from mineral thickeners of silicate type, more preferably from hectorites.

When they are present, the fatty-phase thickeners are present in a content ranging from 0.05% to 10% by weight and preferably from 0.075% to 5% by weight relative to the total weight of the composition.

According to a preferred embodiment, the fatty phase comprises a mineral thickener in a content of greater than 0.05% by weight relative to the total weight of the composition.

The fatty phase of the composition may also comprise any usual liposoluble or lipodispersible additive, for instance other solid or pasty fatty substances such as waxes, fatty alcohols, fatty acids or solvents.

It may also include compounds such as alkylene carbonates such as propylene carbonate, which can enhance the effectiveness of certain fatty-phase thickeners such as silicates.

The amount of fatty phase may range from 0.5% to 50% by weight, preferably from 0.7% to 30% by weight and better still from 1% to 20% by weight, relative to the total weight of the composition.

As mentioned previously, the composition according to the invention comprises an aqueous phase.

The aqueous phase of the composition according to the invention comprises at least water.

The amount of water may represent at least 30% by weight, preferably at least 50% by weight and better still at least 60% by weight relative to the total weight of the composition.

The amount of water may represent from 30% to 98% by weight, preferably from 50% to 95% by weight and better still from 60% to 92% by weight relative to the total weight of the composition.

Preferably, the weight ratio of the amount of water to the amount of oil(s) in the compositions of the invention ranges from 1 to 80, better still from 5 to 70 and even better still from 10 to 60.

The aqueous phase of the composition according to the invention also comprises an aqueous-phase thickener.

According to the present invention, the term “aqueous-phase thickener” means compounds which, by their presence, increase the viscosity of the aqueous phase into which they are introduced by at least 20 cps and preferably by at least 50 cps, at 25° C. and at a shear rate of 1 s⁻¹ (the viscosity may be measured using a cone/plate viscometer, a Haake R600 rheometer or the like).

Aqueous-phase thickeners that may be mentioned include non-associative thickening polymers bearing sugar units.

For the purposes of the present invention, the term “sugar unit” means a unit derived from a carbohydrate of formula Cₙ(H₂O)ₙ₋₁ or (CH₂O)ₙ, which may be optionally modified by substitution and/or by oxidation and/or by dehydration.

The sugar units that may be included in the composition of the thickening polymers of the invention are preferably derived from the following sugars:
glucose;
galactose;
arabinose;
rhamnose;
mannose;
xylose;
fucose;
anhydrogalactose;
galacturonic acid;
glucuronic acid;
mannuronic acid;
galactose sulfate;
anhydrogalactose sulfate and
fructose.

Thickening polymers of the invention that may especially be mentioned include native gums such as:

a) tree or shrub exudates, including:
gum arabic (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (or tragacanth) (polymer of galacturonic acid, galactose, fucose, xylose and arabinose);
   b) gums derived from algae, including:
agar (polymer derived from galactose and anhydrogalactose);
alginates (polymers of mannuronic acid and glucuronic acid);
carrageenans and furcellerans (polymers of galactose sulfate and anhydrogalactose sulfate);
   c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose);
   d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);
   e) plant extracts, including:
cellulose (glucose polymer);
starch (glucose polymer) and
inulin.

These polymers may be physically or chemically modified. A physical treatment that may especially be mentioned is the temperature.

Chemical treatments that may be mentioned include esterification, etherification, amidation or oxidation reactions. These treatments can lead to polymers that may especially be nonionic, anionic or amphoteric.

Preferably, these chemical or physical treatments are applied to guar gums, locust bean gums, starches and celluloses.

The nonionic guar gums that may be used according to the invention may be modified with C₁-C₆ (poly)hydroxyalkyl groups.

Among the C₁-C₆ (poly)hydroxyalkyl groups that may be mentioned, for example, are hydroxymethyl, hydroxyethyl, hydroxypropyl and hydroxybutyl groups.

These guar gums are well known in the prior art and can be prepared, for example, by reacting the corresponding alkene oxides, for instance propylene oxides, with the guar gum so as to obtain a guar gum modified with hydroxypropyl groups.

The degree of hydroxyalkylation preferably ranges from 0.4 to 1.2, and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The botanical origin of the starch molecules used in the present invention may be cereals or tubers. Thus, the starches are chosen, for example, from corn starch, rice starch, cassava starch, barley starch, potato starch, wheat starch, sorghum starch and pea starch.

The starches may be chemically or physically modified especially by one or more of the following reactions: pregelatinization, oxidation, crosslinking, esterification, etherification, amidation, heat treatments.

Distarch phosphates or compounds rich in distarch phosphate will preferentially be used, for instance the products sold under the references Prejel VA-70-T AGGL (gelatinized hydroxypropyl cassava distarch phosphate), Prejel TK1 (gelatinized cassava distarch phosphate) and Prejel 200 (gelatinized acetyl cassava distarch phosphate) by the company Avebe, or Structure Zea from National Starch (gelatinized corn distarch phosphate).

According to the invention, amphoteric starches may also be used, these amphoteric starches comprising one or more anionic groups and one or more cationic groups. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The starch molecules may be derived from any plant source of starch, especially such as corn, potato, oat, rice, tapioca, sorghum, barley or wheat. It is also possible to use the starch hydrolysates mentioned above. The starch is preferably derived from potato.

The non-associative thickening polymers of the invention may be cellulose-based polymers not comprising a C₁₀-C₃₀ fatty chain in their structure.

According to the invention, the term “cellulose-based” polymer means any polysaccharide compound bearing in its structure sequences of glucose residues linked via β-1,4 bonds; besides unsubstituted celluloses, the cellulose derivatives may be anionic, cationic, amphoteric or nonionic.

Thus, the cellulose-based polymers of the invention may be chosen from unsubstituted celluloses, including those in a microcrystalline form, and cellulose ethers.

Among these cellulose-based polymers, cellulose ethers, cellulose esters and cellulose ester ethers are distinguished.

Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the nonionic cellulose ethers without a C₁₀-C₃₀ fatty chain, i.e. which are “non-associative”, mention may be made of (C₁-C₄)alkylcelluloses, such as methylcelluloses and ethylcelluloses (for example, Ethocel standard 100 Premium from Dow Chemical); (poly)hydroxy(C₁-C₄)alkylcelluloses, such as hydroxymethylcelluloses, hydroxyethylcelluloses (for example, Natrosol 250 HHR provided by Aqualon) and hydroxypropylcelluloses (for example, Klucel EF from Aqualon); mixed (poly)hydroxy(C₁-C₄)alkyl-(C₁-C₄)alkylcelluloses, such as hydroxypropylmethylcelluloses (for example, Methocel E4M from Dow Chemical), hydroxyethylmethylcelluloses, hydroxyethylethylcelluloses (for example, Bermocoll E 481 FQ from Akzo Nobel) and hydroxybutylmethylcelluloses.

Among the anionic cellulose ethers without a fatty chain, mention may be made of (poly)carboxy(C₁-C₄)alkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and the sodium salts thereof.

Among the cationic cellulose ethers without a fatty chain, mention may be made of cationic cellulose derivatives such as cellulose copolymers or cellulose derivatives grafted with a water-soluble quaternary ammonium monomer, and described in particular in U.S. Pat. No. 4,131,576, such as (poly)hydroxy(C₁-C₄)alkyl celluloses, for instance hydroxymethyl-, hydroxyethyl- or hydroxypropylcelluloses grafted in particular with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

Among the nonassociative thickening polymers not bearing sugar units that may be used, mention may be made of crosslinked acrylic or methacrylic acid homopolymers or copolymers, crosslinked 2-acrylamido-2-methylpropanesulfonic acid homopolymers and crosslinked acrylamide copolymers thereof, ammonium acrylate homopolymers, or copolymers of ammonium acrylate and of acrylamide, alone or mixtures thereof.

A first family of nonassociative thickening polymers that is suitable for use is represented by crosslinked acrylic acid homopolymers.

Among the homopolymers of this type, mention may be made of those crosslinked with an allyl alcohol ether of the sugar series, for instance the products sold under the names Carbopol 980, 981, 954, 2984 and 5984 by the company Noveon or the products sold under the names Synthalen M and Synthalen K by the company 3 VSA.

The nonassociative thickening polymers may also be crosslinked (meth)acrylic acid copolymers, such as the polymer sold under the name Aqua SF1 by the company Noveon.

The nonassociative thickening polymers may be chosen from crosslinked 2-acrylamido-2-methylpropanesulfonic acid homopolymers and the crosslinked acrylamide copolymers thereof.

Among the partially or totally neutralized crosslinked copolymers of 2-acrylamido-2-methylpropanesulfonic acid and of acrylamide, mention may be made in particular of the product described in Example 1 of document EP 503 853, and reference may be made to said document as regards these polymers.

The composition may similarly comprise, as nonassociative thickening polymers, ammonium acrylate homopolymers or copolymers of ammonium acrylate and of acrylamide.

Among the ammonium acrylate homopolymers that may be mentioned is the product sold under the name Microsap PAS 5193 by the company Hoechst. Among the copolymers of ammonium acrylate and of acrylamide that may be mentioned is the product sold under the name Bozepol C Nouveau or the product PAS 5193 sold by the company Hoechst. Reference may be made especially to documents FR 2 416 723, U.S. Pat. No. 2,798,053 and U.S. Pat. No. 2,923,692 as regards the description and preparation of such compounds.

Cationic thickening polymers of acrylic type can also be used.

Among the aqueous-phase thickening polymers, mention may also be made of the associative polymers that are well known to a person skilled in the art and especially of nonionic, anionic, cationic or amphoteric nature.

It is recalled that “associative polymers” are polymers that are capable, in an aqueous medium, of reversibly associating with each other or with other molecules. Their chemical structure more particularly comprises at least one hydrophilic region and at least one hydrophobic region.

The term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative polymers of anionic type that may be mentioned are:

(a) those comprising at least one hydrophilic unit and at least one fatty-chain allyl ether unit, more particularly those whose hydrophilic unit is formed by an ethylenic unsaturated anionic monomer, more particularly by a vinylcarboxylic acid and most particularly by an acrylic acid or a methacrylic acid or mixtures thereof.

Among these anionic associative polymers, those that are particularly preferred according to the invention are polymers formed from 20% to 60% by weight of acrylic acid and/or of methacrylic acid, from 5% to 60% by weight of lower alkyl(meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl(meth)acrylate, divinylbenzene, (poly)ethylene glycol dimethacrylate and methylenebisacrylamide.

Among the latter polymers, those most particularly preferred are crosslinked terpolymers of methacrylic acid, of ethyl acrylate and of polyethylene glycol (10 EO) stearyl alcohol ether (Steareth 10), in particular those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10).

(b) those comprising i) at least one hydrophilic unit of unsaturated olefinic carboxylic acid type, and ii) at least one hydrophobic unit of the type such as a (C₁₀-C₃₀) alkyl ester of an unsaturated carboxylic acid.

(C₁₀-C₃₀) alkyl esters of unsaturated carboxylic acids that are useful in the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. No. 3,915,921 and U.S. Pat. No. 4,509,949.

Among anionic associative polymers of this type that will be used more particularly are those consisting of from 95% to 60% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those consisting of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among the said above polymers, those most particularly preferred according to the present invention are the products sold by the company Goodrich under the trade names Pemulen TR1®, Pemulen TR2® and Carbopol 1382®, and even more preferentially Pemulen TR1®, and the product sold by the company SEPPIC under the name Coatex SX®.

Mention may also be made of the acrylic acid/lauryl methacrylate/vinylpyrrolidone terpolymer sold under the name Acrylidone LM by the company ISP.

(c) maleic anhydride/C₃₀-C₃₈ α-olefin/alkyl maleate terpolymers, such as the product (maleic anhydride/C₃₀-C₃₈ α-olefin/isopropyl maleate copolymers) sold under the name Performa V 1608® by the company Newphase Technologies.

(d) acrylic terpolymers comprising:
i) about 20% to 70% by weight of an α,β-monoethylenically unsaturated carboxylic acid [A],
ii) about 20% to 80% by weight of a non-surfactant monomer containing α,β-monoethylenic unsaturation other than [A],
iii) about 0.5% to 60% by weight of a nonionic monourethane which is the product of reaction of a monohydric surfactant with a monoisocyanate containing monoethylenic unsaturation,
   such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.

(e) copolymers comprising among their monomers a carboxylic acid containing α,β-monoethylenic unsaturation and an ester of a carboxylic acid containing α,β-monoethylenic unsaturation and of an oxyalkylenated fatty alcohol.

Preferentially, these compounds also comprise as monomer an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer; and also Aculyn 88, also sold by the company Röhm & Haas.

(f) amphiphilic polymers comprising at least one ethylenically unsaturated monomer bearing a sulfonic group, in free or partially or totally neutralized form and comprising at least one hydrophobic part. These polymers may be crosslinked or non-crosslinked. They are preferably crosslinked.

The ethylenically unsaturated monomers bearing a sulfonic group are especially chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof.

(Meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, will more preferentially be used.

2-Acrylamido-2-methylpropanesulfonic acid (AMPS), and also partially or totally neutralized forms thereof, will more particularly be used.

The polymers of this family may be chosen especially from random amphiphilic AMPS polymers modified by reaction with a C₆-C₂₂ n-monoalkylamine or di-n-alkylamine, and such as those described in patent application WO 00/31154. These polymers may also contain other ethylenically unsaturated hydrophilic monomers selected, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid and maleic acid, or mixtures of these compounds.

The preferred polymers of this family are chosen from amphiphilic copolymers of AMPS and of at least one ethylenically unsaturated hydrophobic monomer.

These same copolymers may also contain one or more ethylenically unsaturated monomers not comprising a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid and maleic acid, or mixtures of these compounds.

These copolymers are described especially in patent application EP-A-750 899, U.S. Pat. No. 5,089,578 and in the following Yotaro Morishima publications:
“Self-assembling amphiphilic polyelectrolytes and their nanostructures —Chinese Journal of Polymer Science, Vol. 18, No. 40, (2000), 323-336”;
“Micelle formation of random copolymers of sodium 2-(acrylamido)-2-methylpropanesulfonate and a nonionic surfactant macromonomer in water as studied by fluorescence and dynamic light scattering—Macromolecules, Vol. 33, No. 10, (2000), 3694-3704”;
“Solution properties of micelle networks formed by nonionic moieties covalently bound to a polyelectrolyte: salt effects on rheological behavior—Langmuir, Vol. 16, No. 12, (2000) 5324-5332”;
“Stimuli responsive amphiphilic copolymers of sodium 2-(acrylamido)-2-methylpropanesulfonate and associative macromonomers—Polym. Preprint, Div. Polym. Chem., 40(2), (1999), 220-221”.

Among these polymers, mention may be made of:
crosslinked or non-crosslinked, neutralized or non-neutralized copolymers, comprising from 15% to 60% by weight of AMPS units and from 40% to 85% by weight of (C₈-C₁₆)alkyl(meth)acrylamide or (C₈-C₁₆)alkyl(meth)acrylate units relative to the polymer, such as those described in patent application EP-A750 899;
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Among the cationic associative polymers that may be mentioned are:

(I) cationic associative polyurethanes;

(II) the compound sold by the company Noveon under the name Aqua CC and which corresponds to the INCI name Polyacrylate-1 Crosspolymer.

Polyacrylate-1 Crosspolymer is the product of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

(III) quaternized (poly)hydroxyethylcelluloses modified with groups comprising at least one fatty chain, such as alkyl, arylalkyl or alkylaryl groups comprising at least 8 carbon atoms, or mixtures thereof. The alkyl radicals borne by the above quaternized celluloses or hydroxyethylcelluloses preferably comprise from 8 to 30 carbon atoms.

The aryl radicals preferably denote phenyl, benzyl, naphthyl or anthryl groups. Examples of quaternized alkylhydroxyethylcelluloses containing C₈-C₃₀ fatty chains that may be indicated include the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by the company Aqualon, and the products Crodacel QM®, Crodacel QL® (C₁₂ alkyl) and Crodacel QS® (C₁₈ alkyl) sold by the company Croda and the product Softcat SL 100® sold by the company Aqualon.

(IV) cationic polyvinyllactam polymers.

Such polymers are described, for example, in patent application WO-00/68282.

As cationic poly(vinyllactam) polymers according to the invention, vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers, vinylpyrrolidone/dimethylaminopropylmethacrylamide/lauryldimethylmethacrylamidopropylammonium tosylate or chloride terpolymers are used in particular.

The amphoteric associative polymers are preferably chosen from those comprising at least one non-cyclic cationic unit. Even more particularly, the ones that are preferred are those prepared from or comprising 1 mol % to 20 mol %, preferably 1.5 mol % to 15 mol % and even more particularly 1.5 mol % to 6 mol % of fatty-chain monomer relative to the total number of moles of monomers.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type that may be used according to the invention are preferably chosen from:

(a) copolymers of vinylpyrrolidone and of fatty-chain hydrophobic monomers, of which examples that may be mentioned include:
the products Antaron V216® or Ganex V216® (vinylpyrrolidone/hexadecene copolymer) sold by the company I.S.P.,
the products Antaron V220® or Ganex V220® (vinylpyrrolidone/eicosene copolymer) sold by the company I.S.P.,

(b) copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name Antil 208®,

(c) copolymers of hydrophilic methacrylates or acrylates and of hydrophobic monomers comprising at least one fatty chain, for instance the polyethylene glycol methacrylate/lauryl methacrylate copolymer,

(d) polyurethane polyethers comprising in their chain both hydrophilic blocks usually of polyoxyethylenated nature and hydrophobic blocks, which may be aliphatic sequences alone and/or cycloaliphatic and/or aromatic sequences,

(e) polymers with an aminoplast ether backbone containing at least one fatty chain, such as the Pure Thix® compounds sold by the company Sud-Chemie,

(f) celluloses or derivatives thereof, modified with groups comprising at least one fatty chain, such as alkyl, arylalkyl or alkylaryl groups or mixtures thereof in which the alkyl groups are of C₈, and in particular:

nonionic alkylhydroxyethylcelluloses such as the products Natrosol Plus Grade 330 CS and Polysurf 67 (C₁₆ alkyl) sold by the company Aqualon;

nonionic nonoxynylhydroxyethylcelluloses such as the product Amercell HM-1500 sold by the company Amerchol;

nonionic alkylcelluloses such as the product Bermocoll EHM 100 sold by the company Berol Nobel;

(g) associative guar derivatives, for instance hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

Preferably, the polyurethane polyethers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be envisaged. In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The nonionic fatty-chain polyurethane polyethers may be triblock copolymers in which the hydrophilic block is a polyoxyethylenated chain comprising from 50 to 1000 oxyethylene groups. The nonionic polyurethane polyethers comprise a urethane bond between the hydrophilic blocks, whence arises the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂₋₁₄ alkyl chain, and the product Elfacos T212® containing a C₁₈ alkyl chain, from Akzo.

The product DW 1206B® from Röhm & Haas containing a C₂₀ alkyl chain and a urethane bond, sold at a solids content of 20% in water, may also be used.

Use may also be made of solutions or dispersions of these polymers, especially in water or in aqueous-alcoholic medium. Examples of such polymers that may be mentioned are Rheolate® 255, Rheolate® 278 and Rheolate® 244 sold by the company Rheox. The products DW 1206F and DW 1206J sold by the company Röhm & Haas may also be used.

The polyurethane polyethers that may be used according to the invention are in particular those described in the article by G. Fonnum, J. Bakke and Fk. Hansen —Colloid Polym. Sci., 271, 380-389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold in particular by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the aqueous-phase thickener(s) are chosen from polymers not comprising any sugar units.

More preferentially, the aqueous-phase thickener(s) are chosen from associative or non-associative polymers bearing acrylic or methacrylic units.

The aqueous-phase thickener is preferably present in a content ranging from 0.1% to 20%, more preferentially in an amount ranging from 0.2% to 15% and better still in an amount ranging from 0.5% to 10% by weight, relative to the total weight of the composition.

The aqueous phase may comprise at least one hydrophilic organic solvent, for instance substantially linear or branched lower monoalcohols containing from 1 to 8 carbon atoms, such as ethanol, propanol, butanol, isopropanol or isobutanol; polyols, such as propylene glycol, isoprene glycol, butylene glycol, glycerol, sorbitol, polyethylene glycols and derivatives thereof; and mixtures thereof.

The aqueous phase may also comprise a fixing polymer, other than the aqueous-phase thickeners of the invention.

For the purposes of the present invention, the term “fixing polymer” means any polymer that is capable, by application to the hair, of giving a shape to a head of hair or of holding the hair in an already acquired shape.

The fixing polymer(s) used are selected from ionic, especially anionic, cationic, amphoteric, and nonionic fixing polymers, and mixtures thereof.

Anionic polymers include polymers containing groups derived from carboxylic, sulfonic or phosphoric acids, and having a number-average molecular mass of between 500 and 5 000 000.

The carboxylic groups are provided by unsaturated monocarboxylic or dicarboxylic acid monomers, such as those conforming to the formula:

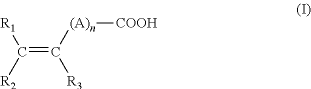

in which n is an integer from 0 to 10, A denotes a methylene group which is optionally connected to the carbon atom of the unsaturated group or to the neighbouring methylene group when n is greater than 1, via a heteroatom such as oxygen or sulfur, R₁ denotes a hydrogen atom or a phenyl or benzyl group, R₂ denotes a hydrogen atom, an alkyl group containing 1 to 4 carbon atoms, or a carboxyl group, R₃ denotes a hydrogen atom, an alkyl group containing 1 to 4 carbon atoms, or a —CH₂—COOH, phenyl or benzyl group.

In the formula (I) above, the alkyl group containing 1 to 4 carbon atoms may more particularly denote methyl and ethyl groups.

The anionic fixing polymers containing carboxylic or sulfonic groups that are preferred are:

A) copolymers of acrylic or methacrylic acid or salts thereof and of acrylamide.

B) Copolymers of acrylic or methacrylic acids with a monoethylenic monomer such as ethylene, styrene, vinyl esters, optionally hydroxylated acrylic or methacrylic acid esters, which are optionally grafted on a polyalkylene glycol such as polyethylene glycol, and are optionally crosslinked. Such polymers are described in particular in French patent 1 222 944 and German patent application No. 2 330 956, the copolymers of this type comprising an optionally N-alkylated and/or hydroxyalkylated acrylamide unit in their chain as described especially in Luxembourg patent applications 75370 and 75371. Mention may also be made of copolymers of acrylic acid and of a C₁-C₄ alkyl methacrylate, methacrylic acid/acrylic acid/ethyl acrylate/methyl methacrylate copolymers, in particular Amerhold DR 25 sold by the company Amerchol, methacrylic acid/ethyl acrylate copolymers, especially as an aqueous dispersion, such as Luviflex Soft and Luvimer MAE sold by the company BASF, and copolymers based on hydroxy esters such as Acudyne 180 from the company Röhm & Haas.

As another anionic fixing polymer from this class, mention may also be made of the branched sequenced anionic butyl acrylate/acrylic acid/methacrylic acid polymer sold under the name Fixate G-100 L by the company Lubrizol (INCI name AMP-Acrylates/Allyl Methacrylate Copolymer).

C) Copolymers derived from crotonic acid, such as those including in their chain vinyl propionate or acetate units, and optionally other monomers such as allyl or methallyl esters, vinyl ethers or vinyl esters of a linear or branched, saturated carboxylic acid with a long hydrocarbon-based chain, such as those containing at least 5 carbon atoms, it being possible optionally for these polymers to be grafted and crosslinked, or else a vinyl, allyl or methallyl ester of an α- or β-cyclic carboxylic acid. Such polymers are described, inter alia, in French patents Nos. 1 222 944, 1 580 545, 2 265 782, 2 265 781, 1 564 110 and 2 439 798. Commercial products that fall within this category are the resins 28-29-30, 26-13-14 and 28-13-10 sold by the company National Starch.

Mention may also be made, as copolymer derived from crotonic acid, of crotonic acid/vinyl acetate/vinyl tert-butylbenzoate terpolymers, and more particularly Mexomer PW supplied by the company Chimex.

D) Polymers derived from maleic, fumaric and/or itaconic acids or anhydrides with vinyl esters, vinyl ethers, vinyl halides, phenylvinyl derivatives, acrylic acid and its esters; these polymers may be esterified. Polymers of these kinds are described in particular in patents U.S. Pat. Nos. 2,047,398, 2,723,248 and 2,102,113, and in patent GB 839 805, and in particular are those sold under the names Gantrez® AN or ES by the company ISP.

Polymers also falling within this category are the copolymers of maleic, citraconic and/or itaconic anhydrides and of an allylic or methallylic ester optionally comprising an acrylamide or methacrylamide group, an α-olefin, acrylic or methacrylic esters, acrylic or methacrylic acids or vinylpyrrolidone in their chain, the anhydride functions being monoesterified or monoamidated. These polymers are described, for example, in French patents 2 350 384 and 2 357 241 by the Applicant.

E) Polyacrylamides comprising carboxylate groups.

F) Polymers comprising sulfonic groups. These polymers may be polymers comprising vinylsulfonic, styrenesulfonic, naphthalenesulfonic, acrylamidoalkylsulfonic or sulfoisophthalate units.

These polymers can in particular be chosen from:
polyvinylsulfonic acid salts with a molecular mass of between about 1000 and 100 000, and also copolymers with an unsaturated comonomer such as acrylic or methacrylic acids and esters thereof, and also acrylamide or derivatives thereof, vinyl ethers and vinylpyrrolidone;
polystyrenesulfonic acid salts, sodium salts, with a molecular mass of about 500 000 and of about 100 000. These compounds are described in patent FR 2 198 719;
polyacrylamidesulfonic acid salts such as those mentioned in patent U.S. Pat. No. 4,128,631.

G) Grafted anionic silicone polymers.

The grafted silicone polymers used are preferably chosen from polymers containing a non-silicone organic backbone grafted with monomers containing a polysiloxane, polymers containing a polysiloxane backbone grafted with non-silicone organic monomers, and mixtures thereof.

H) Anionic polyurethanes, possibly comprising silicone grafts and silicones containing hydrocarbon-based grafts.

Examples of fixing polyurethanes include, in particular, the dimethylolpropionic acid/isophorone diisocyanate/neopentyl glycol/polyesterdiol copolymer (also known under the name polyurethane-1, INCI nomenclature) sold under the brand name Luviset® PUR by the company BASF, and the dimethylolpropionic acid/isophorone diisocyanate/neopentyl glycol/polyesterdiol/silicone diamine copolymer (also known under the name polyurethane-6, INCI nomenclature) sold under the brand name Luviset® Si PUR A by the company BASF.

Another anionic polyurethane that can also be used is Avalure UR 450.

It is also possible to use polymers containing sulfoisophthalate groups, such as the polymers AQ55 and AQ48 sold by the company Eastman.

According to the invention, the anionic polymers are preferably selected from copolymers of acrylic acid such as the acrylic acid/ethyl acrylate/N-tert-butylacrylamide terpolymers sold under the name Ultrahold Strong® by the company BASF, and methacrylic acid/ethyl acrylate copolymers, especially in aqueous dispersion, such as Luviflex Soft and Luvimer MAE, which are sold by the company BASF. Copolymers derived from crotonic acid such as vinyl acetate/vinyl tert-butylbenzoate/crotonic acid terpolymers and crotonic acid/vinyl acetate/vinyl neododecanoate terpolymers, which are sold under the name Resin 28-29-30 by the company National Starch; polymers derived from maleic, fumaric and/or itaconic acids or anhydrides with vinyl esters, vinyl ethers, vinyl halides, phenylvinyl derivatives, acrylic acid and its esters, such as the monoesterified maleic anhydride/methyl vinyl ether copolymer sold under the name Gantrez® ES 425 by the company ISP, Luviset SI PUR, Mexomer PW, elastomeric or non-elastomeric anionic polyurethanes, and polymers containing sulfoisophthalate groups.

The cationic fixing polymers that can be used according to the present invention are preferably chosen from polymers comprising primary, secondary, tertiary and/or quaternary amine groups forming part of the polymer chain or directly attached thereto, and having a molecular weight of between 500 and approximately 5 000 000 and preferably between 1000 and 3 000 000.

Among these polymers, mention may be made more particularly of the following cationic polymers:

(1) Homopolymers or copolymers derived from acrylic or methacrylic esters or amides and containing at least one of the units of formulae below:

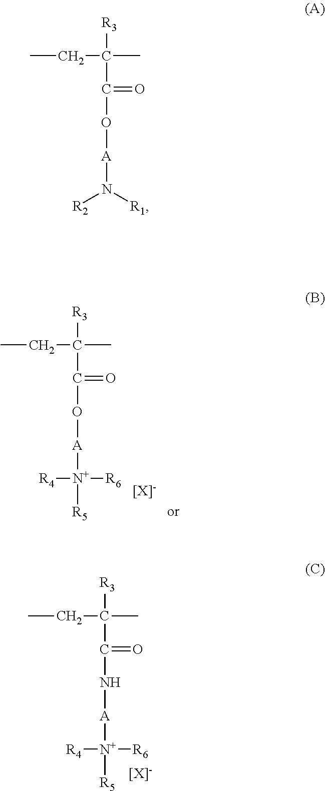

in which:

R₃ denotes a hydrogen atom or a CH₃ group;

A is a linear or branched alkyl group containing from 1 to 6 carbon atoms, or a hydroxyalkyl group containing from 1 to 4 carbon atoms;

R₄, R₅ and R₆, which are identical or different, represent an alkyl group having from 1 to 18 carbon atoms, or a benzyl group;

R₁ and R₂, which are identical or different, each represent a hydrogen atom or an alkyl group having from 1 to 6 carbon atoms;

X denotes a methosulfate anion or a halide such as chloride or bromide.

The copolymers of class (1) further contain one or more units deriving from comonomers which may be selected from the class of acrylamides, methacrylamides, diacetone acrylamides, acrylamides and methacrylamides substituted on the nitrogen by C₁-C₄ alkyl groups, groups derived from acrylic or methacrylic acids or their esters, vinyl lactams such as vinylpyrrolidone or vinylcaprolactam, and vinyl esters.

Accordingly, these copolymers of class (1) may include the following:
copolymers of acrylamide and dimethylaminoethyl methacrylate quaternized with dimethyl sulfate or with a dimethyl halide, such as that sold under the name Hercofloc® by the company Hercules,
copolymers of acrylamide and methacryloyloxyethyltrimethylammonium chloride, described for example in patent application EP-A-080976 and sold under the name Bina Quat P 100 by the company Ciba Geigy,
the copolymer of acrylamide and methacryloyloxyethyltrimethylammonium methosulfate, such as that sold under the name Reten by the company Hercules,
quaternized or non-quaternized vinylpyrrolidone/dialkylaminoalkyl acrylate or methacrylate copolymers, such as the products sold under the name Gafquat® by the company ISP, for instance, Gafquat® 734 or Gafquat® 755, or alternatively the products known as Copolymer® 845, 958 and 937. These polymers are described in detail in French patents 2 077 143 and 2 393 573,
polymers containing a fatty chain and a vinylpyrrolidone unit, such as the products sold under the names Styleze W20 and Styleze W10 by the company ISP,
dimethylaminoethyl methacrylate/vinylcaprolactam/vinylpyrrolidone terpolymers, such as the product sold under the name Gaffix VC 713 by the company ISP, and
quaternized vinylpyrrolidone/dimethylaminopropylmethacrylamide copolymers, such as the products sold under the name Gafquat® HS 100 by the company ISP;

(2) cationic guar gums, preferably containing quaternary ammonium, such as those described in U.S. Pat. Nos. 3,589,578 and 4,031,307, such as guar gums containing trialkylammonium cationic groups. Such products are sold in particular under the trade names Jaguar C13 S, Jaguar C 15 and Jaguar C 17 by the company Meyhall;

(3) quaternary copolymers of vinylpyrrolidone and vinylimidazole;

(4) chitosans or salts thereof; the salts which can be used are more particularly the acetate, lactate, glutamate, gluconate or pyrrolidone carboxylate of chitosan.

Among these compounds, mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol;

(5) cationic cellulose derivatives such as copolymers of cellulose or of cellulose derivatives that are grafted with a water-soluble monomer containing a quaternary ammonium, and are described in particular in patent U.S. Pat. No. 4,131,576, such as hydroxyalkylcelluloses, such as hydroxymethyl-, hydroxyethyl- or hydroxypropylcelluloses grafted in particular with a methacryloyloxyethyltrimethylammonium, methacrylamidopropyl trimethylammonium or dimethyldiallylammonium salt.

The commercialized products which meet this definition are more particularly the products sold under the names Celquat L 200 and Celquat H 100 by the National Starch company.

The amphoteric fixing polymers that can be used in accordance with the invention can be chosen from polymers comprising units B and C distributed randomly in the polymer chain, in which B denotes a unit deriving from a monomer comprising at least one basic nitrogen atom and C denotes a unit deriving from an acid monomer comprising one or more carboxylic or sulfonic groups, or alternatively B and C can denote groups deriving from carboxybetaine or sulfobetaine zwitterionic monomers; B and C can also denote a cationic polymer chain comprising primary, secondary, tertiary or quaternary amine groups, in which at least one of the amine groups bears a carboxylic or sulfonic group connected via a hydrocarbon-based group, or alternatively B and C form part of a chain of a polymer containing an ethylenedicarboxylic unit in which one of the carboxylic groups has been made to react with a polyamine comprising one or more primary or secondary amine groups.

The amphoteric polymers corresponding to the definition given above that are more particularly preferred are chosen from the following polymers:

1) Polymers resulting from the copolymerization of a monomer derived from a vinyl compound bearing a carboxylic group, such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkyl methacrylamide and acrylamide. Such compounds are described in U.S. Pat. No. 3,836,537.

The vinyl compound may also be a dialkyldiallylammonium salt such as diethyldiallylammonium chloride.

2) Polymers containing units which derive:

a) from at least one monomer selected from acrylamides or methacrylamides which are substituted on the nitrogen by an alkyl group,

b) from at least one acidic comonomer containing one or more reactive carboxylic groups, and

c) from at least one basic comonomer, such as esters with primary, secondary, tertiary and quaternary amine substituents of acrylic and methacrylic acids, and the product of quaternization of dimethylaminoethyl methacrylate with dimethyl or diethyl sulfate.

The N-substituted acrylamides or methacrylamides which are more particularly preferred according to the invention are groups in which the alkyl groups contain from 2 to 12 carbon atoms, and more particularly N-ethylacrylamide, N-tert-butylacrylamide, N-tert-octylacrylamide, N-octylacrylamide, N-decylacrylamide, N-dodecylacrylamide, and also the corresponding methacrylamides.

The acidic comonomers are selected more particularly from acrylic, methacrylic, crotonic, itaconic, maleic and fumaric acids and also the alkyl monoesters having 1 to 4 carbon atoms of maleic or fumaric acid or anhydride. The preferred basic comonomers are aminoethyl, butylaminoethyl, N,N′-dimethylaminoethyl and N-tert-butylaminoethyl methacrylates. The copolymers whose CTFA (4th edition, 1991) name is octylacrylamide/acrylates/butylaminoethyl methacrylate copolymer, such as the products sold under the name Amphomer® or Lovocryl® 47 by the company National Starch, are particularly used.

3) Alkylated and crosslinked polyaminoamides deriving wholly or partly from polyaminoamides of general formula:

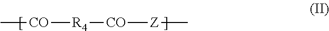

in which R₄ represents a divalent group derived from a saturated dicarboxylic acid, from a mono- or dicarboxylic aliphatic acid with an ethylenic double bond, from an ester of an alcohol containing 1 to 6 carbon atoms with these acids, or from a group deriving from the addition of any one of the said acids with a bis-primary amine or bis-secondary-derived amine, and Z denotes a group of a bis-primary or mono- or bis-secondary polyalkylene-polyamine, and preferably represents:

a) in proportions of 60 mol % to 100 mol %, the group

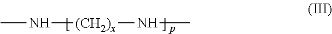

where x=2 and p=2 or 3, or else x=3 and p=2,

this group deriving from diethylenetriamine, triethylenetetramine or dipropylenetriamine;

b) in proportions of 0 to 40 mol %, the group (Ill) above, in which x=2 and p=1, which derives from ethylenediamine, or the group deriving from piperazine;

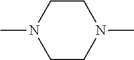

c) in proportions of 0 to 20 mol %, the group —NH—(CH2)6-NH— deriving from hexamethylenediamine, these polyaminoamines being crosslinked by addition of a difunctional crosslinking agent selected from epihalohydrins, diepoxides, dianhydrides, bis-unsaturated derivatives, by means of 0.025 to 0.35 mol of crosslinking agent per amine group of the polyaminoamide, and being alkylated by the action of acrylic acid, chloroacetic acid or an alkane sultone or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond, for instance acrylic acid, methacrylic acid and itaconic acid. The alkane sultones used in the alkylation are preferably propane sultone or butane sultone; the salts of the alkylating agents are preferably the sodium or potassium salts.

4) Polymers containing zwitterionic units of formula:

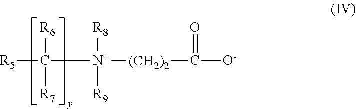

in which R₅ denotes a polymerizable unsaturated group, such as an acrylate, methacrylate, acrylamide or methacrylamide group, y and z each represent an integer from 1 to 3, R₆ et R₇ represent a hydrogen atom or a methyl, ethyl or propyl group, R₈ and R₉ represent a hydrogen atom or an alkyl group such that the sum of the carbon atoms in R₁₀ and R₁₁ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.

5) Polymers derived from chitosan, containing monomer units conforming to the following formulae:

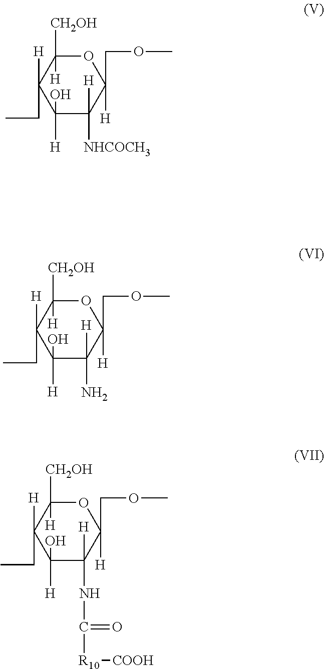

the unit (V) being present in proportions of between 0 and 30%, the unit (VI) in proportions of between 5% and 50% and the unit (VII) in proportions of between 30% and 90%, it being understood that, in this unit F, R₁₀ represents a group of formula:

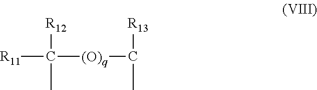

in which, if q=0, R₁₁, R₁₂ and R₁₃, which may be identical or different, each represent a hydrogen atom, a methyl, hydroxyl, acetoxy or amino residue, a monoalkylamine residue or a dialkylamine residue that are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the groups R₁₁, R₁₂ and R₁₃ being, in this case, a hydrogen atom;

or, if q=1, R₁₁, R₁₂ and R₁₃ each represent a hydrogen atom, as well as the salts formed by these compounds with bases or acids.

6) Polymers derived from the N-carboxyalkylation of chitosan.

7) Polymers of units corresponding to the general formula (IX) described, for example, in French patent 1 400 366:

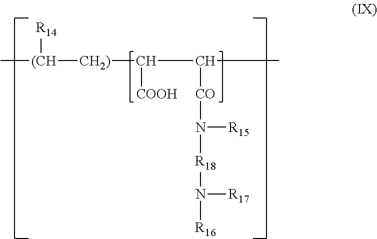

in which R₁₄ represents a hydrogen atom or a CH₃O, CH₃CH₂O, or phenyl group, R₁₅ denotes hydrogen or a C₁₋₄ alkyl group such as methyl and ethyl, R₁₆ denotes hydrogen or a C₁₋₄ alkyl group such as methyl and ethyl, R₁₇ denotes a C₁₋₄ alkyl group such as methyl and ethyl or a group conforming to the formula: —R₁₈—N(R₁₆)₂, with R₁₈ representing a —CH₂—CH₂—, —CH₂—CH₂—CH₂—, or —CH₂—CH(CH₃)— group and R₁₆ having the meanings given above,

and also the higher homologues of these groups, containing up to 6 carbon atoms.

8) Amphoteric polymers of type -D-X-D-X—, selected from:

a) polymers obtained by the action of chloroacetic acid or sodium chloroacetate on compounds comprising at least one unit of formula:

-D-X-D-X-D- (X)

where D denotes a group

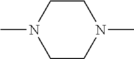

and X denotes the symbol E or E′, where E or E′, which are identical or different, denote a divalent group which is an alkylene group having a straight or branched chain that contains up to 7 carbon atoms in the main chain and is unsubstituted or substituted by hydroxyl groups, and may further comprise oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine and/or alkenylamine groups, hydroxyl, benzylamine, amine oxide, quaternary ammonium, amide, imide, alcohol, ester and/or urethane groups.

b) The polymers of formula:

-D-X-D-X— (XI)

where D denotes a group

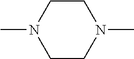

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate.

9) (C1-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers may also comprise other vinyl comonomers such as vinylcaprolactam.

According to a preferred embodiment of the invention, the amphoteric fixing polymers that may be used in the aerosol device according to the invention may be chosen from branched block copolymers comprising:

(a) nonionic units derived from at least one monomer chosen from C₁-C₂₀ alkyl(meth)acrylates, N-mono-(C₂-C₁₂ alkyl)-(meth)acrylamides and N,N-di-(C₂-C₁₂ alkyl)-(meth)acrylamides,

(b) anionic units derived from at least one monomer chosen from acrylic acid and methacrylic acid, and

(c) polyfunctional units derived from at least one monomer containing at least two polymerizable unsaturated functional groups,

and preferably having a structure consisting of hydrophobic blocks onto which are fixed, via polyfunctional units (c), several more hydrophilic blocks.

Preferably, the amphoteric polymers have at least two glass transition temperatures (Tg), at least one of which is greater than 20° C. and the other is less than 20° C.

The preferred amphoteric polymers are polymers comprising units derived:

a) from at least one monomer chosen from acrylamides and methacrylamides substituted on the nitrogen with an alkyl group,

b) from at least one acidic comonomer containing one or more reactive carboxylic groups, and

c) from at least one basic comonomer, such as esters with primary, secondary, tertiary and quaternary amine substituents of acrylic and methacrylic acids, and the product of quaternization of dimethylaminoethyl methacrylate with dimethyl or diethyl sulfate.

Mention may be made in particular of the polymers sold under the name Amphomer by the company National Starch.

The nonionic fixing polymers that may be used according to the present invention are chosen, for example, from:
polyalkyloxazolines,
vinyl acetate homopolymers,
vinyl acetate copolymers, for instance copolymers of vinyl acetate and acrylic ester, copolymers of vinyl acetate and ethylene, or copolymers of vinyl acetate and maleic ester, for example dibutyl maleate,
homopolymers and copolymers of acrylic esters, for instance copolymers of alkyl acrylates and alkyl methacrylates, such as the products provided by the company Röhm & Haas under the names Primal® AC-261 K and Eudragit® NE 30 D, by the company BASF under the name 8845, and by the company Hoechst under the name Appretan® N9212,
copolymers of acrylonitrile and a nonionic monomer chosen, for example, from butadiene and alkyl(meth)acrylates, such as the products provided under the name CJ 0601 B by the company Röhm & Haas,
styrene homopolymers,
styrene copolymers, for instance copolymers of styrene and an alkyl(meth)acrylate, such as the products Mowilith® LDM 6911, Mowilith® DM 611 and Mowilith® LDM 6070, which are provided by the company Hoechst, the products Rhodopas® SD 215 and Rhodopas® DS 910, which are provided by the company Rhone Poulenc, copolymers of styrene, alkyl methacrylate and alkyl acrylate, copolymers of styrene and butadiene, or copolymers of styrene, butadiene and vinylpyridine,
polyamides,
vinyllactam homopolymers such as vinylpyrrolidone homopolymers and such as the polyvinylcaprolactam sold under the name Luviskol® Plus by the company BASF,
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers, for instance the product sold under the name Luviskol® VAP 343 by the company BASF, and
poly(vinyl alcohols).

The alkyl groups in the abovementioned nonionic polymers preferably contain from 1 to 6 carbon atoms.

The fixing polymer is preferably an ionic fixing polymer, preferably an anionic fixing polymer.

When they are present, the fixing polymers are preferably present in the composition in an amount ranging from 0.1% to 20% by weight, preferably from 0.5% to 15% by weight and more preferably from 1% to 10% by weight, relative to the total weight of the composition.

Preferably, the composition according to the invention does not comprise any surfactant. When it does comprise the same, the composition according to the invention comprises less than 2% of surfactant.

The amount of aqueous phase may range from 50% to 99.5% by weight, preferably from 60% to 95% by weight and better still from 70% to 90% by weight, relative to the total weight of the composition.

The composition according to the invention may comprise active agents conventionally used in the field of cosmetics, other than those described previously, and chosen from silicones, direct dyes, in particular cationic or natural direct dyes, or oxidation dyes, organic or mineral pigments, UV-screening agents, resins, fragrances, peptizers, vitamins, amino acids, preserving agents, long-lasting hair shaping agents, especially thiolated organic reducing agents, non-thiolated organic reducing agents, alkaline agents, etc.

Needless to say, a person skilled in the art will take care to select the optional additional compounds and/or the amount thereof such that the advantageous properties of the compositions used according to the invention are not, or are not substantially, adversely affected by the envisaged addition.

Preferably, the composition according to the invention does not comprise any superabsorbent polymer, namely a polymer that is capable in its dry form of spontaneously absorbing at least 20 times its own weight of aqueous fluid, in particular of water and especially distilled water.

Preferably, the composition is in the form of gel, namely aqueous thickened solution, which comprises oily inclusions, such as oily volutes. More preferably, the composition is in the form of transparent oily gel with inclusions such as oily volutes. More preferably, the composition is fully gel, both phases being thickened.

Preferably, the composition has a viscosity of greater than or equal to 0.1 Pa·s and better still ranging from 0.1 Pa·s to 500 Pa·s and even better still from 0.5 Pa·s to 300 Pa·s, and even better still from 1 Pa·s to 200 Pa·s at a temperature of 25° C. and at a shear rate of 1 s⁻¹ (measurable, for example, with a Haake RS600 rheometer).

The composition according to the invention may be obtained by mixing the two phases using a static mixer.

In particular, to make the composition according to the invention, the ingredients of the fatty phase are mixed together, on the one hand, and the ingredients of the aqueous phase are mixed together, on the other hand. Each phase is introduced separately into the static mixer, namely a tube inside which is a three-dimensional structure promoting the appearance of turbulence during the passage of a fluid. The phases are mixed by a static device, i.e. a device that is not driven by a rotary system, thus avoiding dispersion of the fatty phase in the aqueous phase, especially in the form of globules. A mixture in which the two phases are visually distinct is obtained.

A subject of the invention is also a composition according to the invention made using a static mixer.

The composition according to the invention may especially be used in leave-in or rinse-out application to the hair.

A subject of the invention is also a cosmetic hair treatment process, which consists in applying to the hair an effective amount of a composition as has just been described, followed by optionally rinsing it out after an optional leave-in time, in the presence or absence of heat.

### EXAMPLES

The examples that follow are given as illustrations of the present invention. In these examples, all the amounts are indicated as weight percentages of active material (AM) relative to the total weight of the composition.

| | Name | A | B |
|---|---|---|---|
| 1 | Avocado oil | 4.63 | 1.89 |
| 2 | Stearalkonium hectorite ⁽¹⁾ | 0.19 | 0.08 |
| 3 | Propylene carbonate | 0.06 | 0.02 |
| 4 | Acrylates/steareth-20 methacrylate | 1.48 | — |
| | crosspolymer ⁽²⁾ | | |
| 5 | Carbomer ⁽³⁾ | — | 1.27 |
| 6 | Acrylates/hydroxy ester acrylates | 2.52 | — |
| | copolymer ⁽⁴⁾ | | |
| 7 | Propylene glycol | 2.85 | — |
| 8 | PEG-40 hydrogenated castor oil | 0.57 | 0.88 |
| 9 | Ethylhexyl glycerol | 0.47 | — |
| 10 | Magnesium gluconate | 0.01 | — |
| 11 | Calcium gluconate | 0.01 | — |
| 12 | Triethanolamine | 1.42 | 1.86 |
| 13 | Yellow 5 | 0.005 | — |
| 14 | Blue 1 | 0.0004 | — |
| 15 | Red 33 | — | 0.0005 |
| 16 | Fragrance | 0.28 | 0.29 |
| 17 | Preserving agent | qs | qs |
| 18 | Water | qs 100 | qs 100 |

⁽¹⁾ sold under the reference Bentone 27 V CG by the company Elementis
⁽²⁾ sold under the reference Aculyn 88 by the company Dow Chemical
⁽³⁾ sold under the reference Synthalen K by the company 3V
⁽⁴⁾ sold under the reference Acudyne 180 by the company Dow Chemical

Composition A, which is a styling gel, is prepared.

Using a static mixer, the oil phase comprising ingredients 1, 2 and 3 is mixed with the gel phase comprising the other ingredients of the composition. The composition obtained is in the form of a translucent green gel containing very aesthetic whitish volutes.

Composition A is applied to the hair by hand in leave-in mode. The surplus product is easy to remove from the hands on rinsing. The composition gives the hair a soft feel and flexible hold.

Composition B, which is a care gel, is prepared.

Using a static mixer, the oil phase comprising ingredients 1, 2 and 3 is mixed with the gel phase comprising the other ingredients of the composition. The composition obtained is in the form of a translucent red gel containing very aesthetic whitish volutes.

Composition B is applied to the hair and gives the hair suppleness and sheen, and also a fresh effect on application.

The following composition C was also realized, all the amounts are indicated as weight percentages of active material (AM) relative to the total weight of the composition.

| | Nom INCI | C |
|---|---|---|
| 1 | PARAFFINUM LIQUIDUM | 2.30 |
| 2 | Octyldodecanol | 2.30 |
| 3 | Disteardimonium hectorite ⁽¹⁾ | 0.30 |
| 4 | Propylene Carbonate | 0.10 |
| 5 | CARBOMER ⁽²⁾ | 1.23 |
| 6 | Caprylyl glycol | 0.38 |
| 7 | PEG-40 hydrogenated castor oil | 0.85 |
| 8 | Triethanolamine | 1.80 |
| 9 | Red 33 | 0.000475 |
| 10 | Fragrance | 0.28 |
| 11 | Preserving agent | qs |
| 12 | Water | Qsp 100 |

⁽¹⁾ sold under the reference BENTONE 38 V CG by ELEMENTIS
⁽²⁾ sold under the reference SYNTHALEN K by 3V

Composition C, which is a care gel, is prepared.

Using a static mixer, the oil phase comprises ingredients 1 to 4 is mixed with the gel phase comprising other ingredients. The composition obtained is in the form of a red marbled translucent gel containing very aesthetic whitish volutes.

The yield stress of composition C is equal to 57 Pa.

Furthermore, the yield stress of the oil phase of composition C is equal to 94 Pa and the yield stress of the aqueous phase of composition C is equal to 53 Pa.

The visual appearance of marble is stable even after several months of storage. The interfaces between the two phases remain sharp. No variation in thickness of the volutes were found. No recovery of the oil phase was observed.

Composition C is applied to hair and brings a good conditioning effect (soft and smooth hair).

1. Cosmetic composition comprising:
a fatty phase comprising at least one oil;
an aqueous phase comprising at least one aqueous-phase thickener, the two phases being visually distinct.

2. Composition according to claim 1, characterized in that the oil(s) are chosen from C₆-C₁₆ lower alkanes; linear or branched hydrocarbons of mineral or synthetic origin containing more than 16 carbon atoms; non-silicone oils of animal origin or oils of plant origin; fluoro oils; liquid fatty alcohols; liquid fatty esters; non-salified liquid fatty acids; silicone oils; or mixtures thereof, and are preferably chosen from C₆-C₁₆ lower alkanes; linear or branched hydrocarbons of mineral or synthetic origin containing more than 16 carbon atoms; liquid fatty alcohols; oils of plant origin; or mixtures thereof.

3. Composition according to any one of the preceding claims, characterized in that the oil(s) are present in an amount ranging from 0.1% to 20%, more preferentially in an amount ranging from 1% to 10% and better still in an amount ranging from 2% to 5% by weight, relative to the total weight of the composition.

4. Composition according to any one of the preceding claims, characterized in that the fatty phase also comprises at least one fatty-phase thickener, preferably chosen from mineral fatty-phase thickeners and organic fatty-phase thickeners.

5. Composition according to the preceding claim, characterized in that the mineral fatty-phase thickeners are chosen from silicates and silicas, preferably from silicates.

6. Composition according to claim 4, characterized in that the organic fatty-phase thickeners are chosen from semicrystalline polymers, non-silicone polyamides, silicone polyamides, monoalkyl or polyalkyl esters of saccharides or of polysaccharides, N-acylamino acid amide derivatives, copolymers comprising one or more alkylene and/or styrene blocks, and elastomeric organopolysiloxanes, and mixtures thereof.

7. Composition according to any one of the preceding claims, characterized in that the fatty-phase thickener is present in a content ranging from 0.05% to 10% by weight relative to the total weight of the composition and preferably from 0.075% to 5% by weight relative to the total weight of the composition.

8. Composition according to any one of the preceding claims, characterized in that the amount of fatty phase ranges from 0.5% to 50% by weight, preferably from 0.7% to 30% by weight and better still from 1% to 20% by weight, relative to the total weight of the composition.

9. Composition according to any one of the preceding claims, characterized in that the aqueous-phase thickener is chosen from non-associative thickening polymers bearing sugar units, non-associative thickening polymers not bearing sugar units and associative thickening polymers.

10. Composition according to any one of the preceding claims, characterized in that the aqueous-phase thickener is chosen from associative or non-associative thickening polymers bearing acrylic or methacrylic units.

11. Composition according to any one of the preceding claims, characterized in that the aqueous-phase thickener is present in an amount ranging from 0.1% to 20%, more preferentially in an amount ranging from 0.2% to 15% and better still in an amount ranging from 0.5% to 10% by weight, relative to the total weight of the composition.

12. Composition according to any one of the preceding claims, characterized in that the amount of aqueous phase ranges from 50% to 99.5% by weight, preferably from 60% to 95% by weight and better still from 70% to 90% by weight, relative to the total weight of the composition.

13. Composition according to any one of the preceding claims, characterized in that it comprises a fixing polymer chosen from nonionic, anionic, cationic and amphoteric fixing polymers, preferably from anionic fixing polymers.

14. Composition according to any one of the preceding claims, characterized in that it comprises less than 2% of surfactant and preferably does not comprise any surfactant.

15. Composition according to any one of the preceding claims, characterized in that it has a yield stress greater than or equal to 0.1 Pa, at 25° C., preferably ranging from 0.1 Pa to 300 Pa, preferably ranging from 1 Pa to 250 Pa, and still preferably from 10 Pa to 200 Pa.

16. Composition according to any one of the preceding claims, characterized in that it has a viscosity of greater than or equal to 0.1 Pa·s and better still ranging from 0.1 Pa·s to 500 Pa·s and even better still from 0.5 Pa·s to 300 Pa·s, and even better still from 1 Pa·s to 200 Pa·s at a temperature of 25° C. and at a shear rate of 1 s⁻¹.

17. Composition according to any one of the preceding claims, characterized in that it is obtained by mixing the two phases using a static mixer.

18. Cosmetic hair treatment process, characterized in that it consists in applying to the hair an effective amount of a composition according to any one of claims 1 to 17 and then in optionally rinsing it out after an optional leave-in time, in the presence or absence of heat.

19. Use of the cosmetic composition according to any one of claims 1 to 17 for caring for the hair and/or for shaping the hair.
